(19) United States
(12) Patent Application Publication
(10) Pub. No.: US 2002/0099336 A1
(43) Pub. Date: Jul. 25, 2002

(54) Slip-proof syringe

(76) Inventors: Lin Wang, Hsi Chih City (TW); Chung-Hsin Yang, San Chung City (TW)

Correspondence Address: JACOBSON HOLMAN PLLC, 400 SEVENTH STREET N.W., SUITE 600, WASHINGTON, DC, 20004, US

(21) Appl. No.: 10/112,371

(22) Filed: Apr. 1, 2002

Related U.S. Application Data
(63) Continuation-in-part of application No. 09/746,482, filed on Dec. 26, 2000.

(51) Int. Cl. A61M 5/32; A61M 25/16

(52) U.S. Cl. 604/198; 604/171; 604/535

(57) ABSTRACT

A syringe has two groups of oppositely formed slip-proof straps axially formed on a periphery of the barrel so that when the user is holding the barrel to pull the recycled needle back into the barrel, the user is able to have a firm grip to the barrel. Further, the plunger of the syringe has a square head formed on a distal end of the plunger and extending out of the barrel so as to facilitate the user to pull the needle.

### CROSS REFERENCE

The is application is a continuation-in-part of the pending application filed on Dec. 26, 2000, with the application Ser. No. 09/746,482 by the same applicant of this application. The content in the pending application is hereinafter incorporated without further description.

### BACKGROUND OF THE INVENTION

1. Field of the Invention

The present invention relates to a syringe, and more particularly to a slip-proof syringe which is able to ensure the user to have a firm grip when holding the syringe. The syringe has two groups of oppositely formed slip-proof straps axially formed on a periphery of the barrel so that when the user is holding the barrel to pull the recycled needle back into the barrel, the user is able to have a firm grip to the barrel. Further, the plunger of the syringe has a square head formed on a distal end of the plunger and extending out of the barrel so as to facilitate the user to pull the needle.

2. Description of Related Art

An ordinary syringe when recycling the needle requires the user to firmly hold the barrel so as that the user is able to carefully detach the needle from the needle hub. Because directly detaching the needle from the needle nub is too dangerous, a new method of recycling the needle is invented, which is described in U.S. application Ser. No. 09/746,482 by Mr. Chung-Hsin Yang. The method described in the application is novel and is safe for the user. However, normally the needle recycling is performed by most the paramedic personnel who usually wears a pair of rubber gloves and the rubber gloves may cause a slip while holding the barrel trying to pull the needle back into the barrel, which is very dangerous for the user. Because this slip may cause the user to be infected by virus.

To overcome the shortcomings, the present invention intends to provide an improved slip-proof syringe to mitigate or obviate the aforementioned problems.

### SUMMARY OF THE INVENTION

The primary objective of the invention is to provide a slip-proof syringe having a pair of oppositely formed slip-proof straps axially formed on a periphery of the barrel so that when the user is holding the barrel to pull the recycled needle back into the barrel, the user is able to have a firm grip to the barrel.

Another objective of the invention is to provide a slip-proof syringe hving a square head formed on a distal end of the plunger and extending out of the barrel so as to facilitate the user to pull the needle.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of the syringe of the present invention;

FIG. 2 is a cross sectional view of the barrel of the syringe in FIG. 1;

FIG. 3 is an end view showing the slip-proof straps oppositely formed on a periphery of the barrel; and

FIG. 4 is an end view showing that the plunger has a square head formed on a distal end of the plunger.

### DETAILED DESCRIPTION OF PREFERRED EMBODIMENT

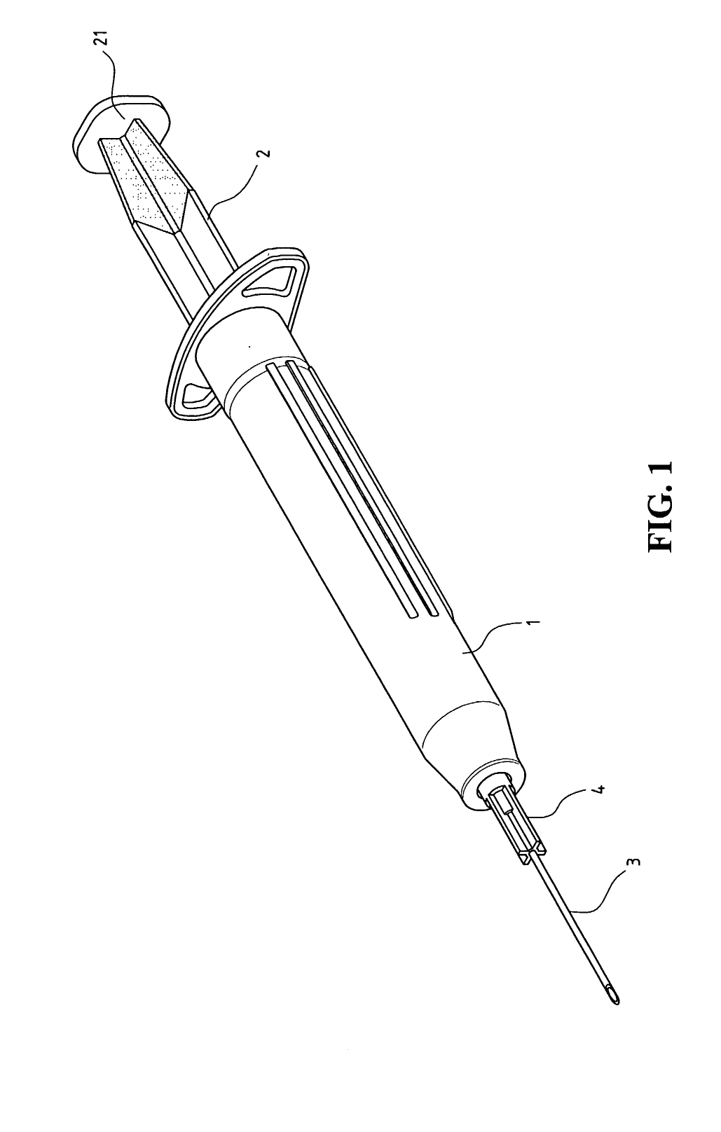

With reference to FIG. 1, the syringe constructed in accordance with the present invention has a barrel 1, a plunger 2 movably received in the barrel 1 and a needle 3 securely mounted on a needle hub 4 which is detachably mounted on a distal end of the barrel 1.

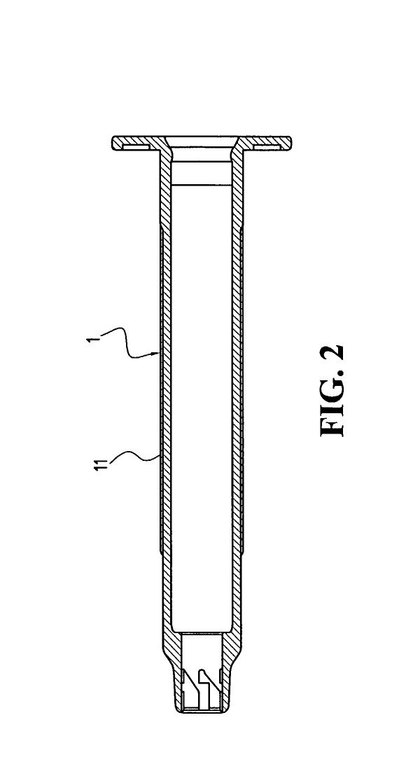

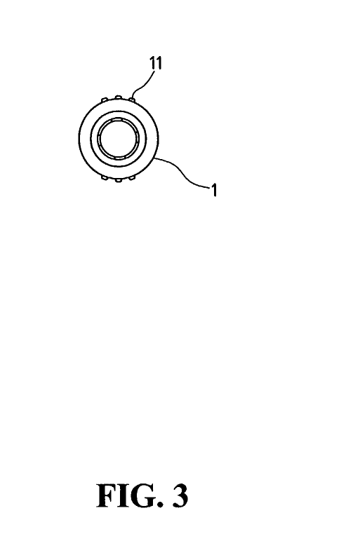

With reference to FIGS. 2 and 3, the barrel 1 has two groups of oppositely formed slip-proof straps 11 axially formed on an outer periphery of the barrel 1 and each group is composed of three slip-proof straps 11. With the slip-proof straps 11 oppositely formed on the periphery of the barrel 1, the user is able to have a firm grip of the barrel 1 when pulling the needle 3 back into the barrel 1.

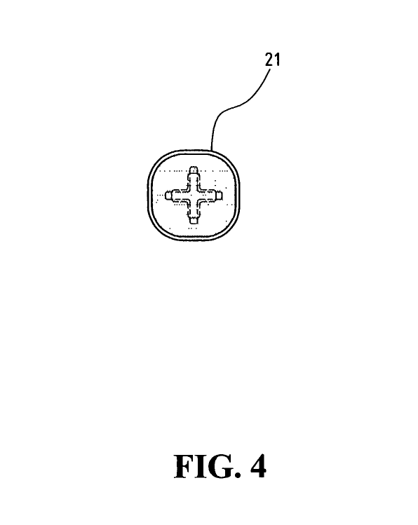

Beside the slip-proof straps 11, the syringe of the invention has a square head 21 formed on a distal free end of the plunger 2, as shown in FIG. 4, so that the user's grip to the plunger 2 is facilitated even the user is wearing a pair of rubber gloves.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. In a slip-proof syringe having a barrel, a plunger movably received in the barrel and a needle securely mounted on a needle hub which is detachably mounted on a distal end of the barrel, wherein the improvements comprise:
the barrel having two groups of oppositely formed slip-proof straps axially formed on an outer periphery of the barrel,
whereby the slip-proof straps are able to assist a firm grip to the barrel when pulling the needle back into the barrel.

2. The syringe as claimed in claim 1, wherein each group has three slip-proof straps.

3. The syringe as claimed in claim 2, wherein the plunger has a square head formed on a free end of the plunger so as to facilitate the holding of the plunger.
